(19) United States
(12) Patent Application Publication
(10) Pub. No.: US 2024/0329019 A1
(43) Pub. Date: Oct. 3, 2024

(54) METHODS FOR THE USE OF NEURAL ACTIVITY PATTERNS FROM THE OLFACTORY SYSTEM OF SERVICE ANIMALS FOR MONITORING OF HEALTH STATES OF LIVING ORGANISMS AND DISEASE BIOMARKER IDENTIFICATION

(71) Applicants: New York University, New York, NY (US); Canaery Inc., Media, PA (US)

(72) Inventors: Dmitry RINBERG, New York, NY (US); Armen ENIKOLOPOV, Media, PA (US); Joshua HARVEY, New York, NY (US)

(73) Assignees: New York University, New York, NY (US); Canaery Inc., Media, PA (US)

(21) Appl. No.: 18/577,667

(22) PCT Filed: Jul. 8, 2022
(86) PCT No.: PCT/US2022/036584

§ 371 Date: Jan. 8, 2024

Related U.S. Application Data
(60) Provisional application No. 63/220,365, filed on Jul. 9, 2021.

(51) Int. Cl. G01N 33/00; G06F 3/01

(52) U.S. Cl. CPC G01N 33/0001; G06F 3/015

(57) ABSTRACT

A method of identification of an odorprint of a health state of a living organism can include monitoring the composition of volatile compounds emitted by the organism using the olfactory system of the service animal equipped with the brain machine interface. The method can include finding the specific signature of the bio-electronic nose signal responsible for identification of a volatile compound odorprint of a health state of an organism. The method can include applying machine learning techniques to multi-component bio-electronic nose signals. The method can include identifying specific volatile compounds or their ratios carrying information about a health state of an organism.

### CROSS-REFERENCE TO RELATED APPLICATIONS

This application claims priority to and the benefit of U.S. Provisional Application No. 63/220,365, filed on Jul. 9, 2021, the content of which is incorporated by reference in its entirety.

### TECHNICAL FIELD

The present disclosure relates generally to the field of diagnostic medicine, brain machine interfaces, and chemical analysis. More particularly, the present disclosure relates to the use of a neural response to odorants to identify health states.

### BACKGROUND

Living organisms (e.g., animals, plants, etc.) can emit volatile compounds (VCs). Changes in health state (e.g., state of an organism's health, health-specific state, etc.) can affect the distribution of the emitted VCs. Disease states, which can be a sub-set of health states, can be associated with a unique VC distribution signature (e.g., odorprint). For humans, reliable readout of information about the health-specific state of the organism can provide a path for non-invasive and non-contact disease diagnostics relevant to medicine and public health. Access to this information for animals or plants can be a crucial tool in agriculture for monitoring the health state of animals or plants. Furthermore, deciphering the VC signatures associated with a specific health state of an organism can enable better understanding of the physiology of healthy or diseased living organisms, and identification of health state biomarkers, which can pave a way to developing alternative diagnostic and pharmacological tools, or other treatment methods.

### SUMMARY

Aspects and advantages of embodiments of the present disclosure will be set forth in part in the following description, or can be learned through practice of the embodiments.

The systems and methods of the present disclosure, in some embodiments, relate to a method of identification of an odorprint of a health state, such as disease, of a living organism (e.g., humans, animals, plants, etc.) by monitoring the composition of volatile compounds emitted by the organism using the olfactory system of the service animal equipped with a brain machine interface (BMI). In some embodiments, the method includes finding the specific signature of a neural signal capable of identification of a volatile compound odorprint of a health state of an organism by presenting a large number of samples from control and experimental subjects, and applying machine learning techniques to extract a characteristic signal. In some embodiments, the method includes identifying specific volatile compounds or their ratios carrying information about a health state of an organism by combining neural signals with a means of separating constituent components in mixtures based on their physicochemical properties, and with a means of elucidating the chemical structure and concentrations of the individual components following separation.

These and other features, aspects, and advantages of various embodiments of the present disclosure will become better understood with reference to the following description and appended claims. The accompanying drawings, which are incorporated in and constitute a part of this specification, illustrate example embodiments of the present disclosure and, together with the description, serve to explain the related principles.

### BRIEF DESCRIPTION OF THE DRAWINGS

The details of one or more implementations of the subject matter described in this specification are set forth in the accompanying drawings and the description below. Other features, aspects, and advantages of the subject matter will become apparent from the description, the drawings, and the claims.

FIG. 1 illustrates a schematic of a brain machine interface, according to an embodiment.

FIG. 2 illustrates a method of identifying an odorprint of a health state of an organism, according to an embodiment.

FIG. 3 illustrates a method of obtaining a signature of a brain machine interface, according to an embodiment.

FIG. 4 illustrates a method of identifying volatile compounds, according to an embodiment.

Like reference numbers and designations in the various drawings indicate like elements.

### DETAILED DESCRIPTION

This disclosure relates generally to methods for identifying an odorprint of a health state, obtaining a signature of a brain machine interface (BMI), and identifying specific volatile compounds or their ratios which carry information about the health state of an organism. The methods described herein relate to uses of the animal olfactory system in combination with the brain machine interface detecting and deciphering the features of the volatile compound distribution carrying information about specific health states, including diseases, of living organisms. The methods described herein can be used for human health state assessment, animal health state assessment, and agricultural monitoring (e.g., monitoring the state of plants in agriculture), as well as for the detection of health states not classified as pathological (e.g. the state of estrous cycle in livestock).

Analytical devices can have limited ability to read the whole spectrum of the volatile compounds (VCs) emitted by living organisms. The highly variable distribution of VCs can prevent efficient identification of the features of this distribution carrying information about a specific disease or health state. Trained animals (e.g., dogs, rats) can be used to detect diseases and non-pathological health states. They can significantly outperform modern analytical methods, mainly due to their higher sensitivity to a large variety of VCs and their ability to filter out an odorprints from highly variable background noise. However, using trained animals is poorly scalable and does not allow for identification of chemicals constituting the health state odorprint.

Therefore, a need exists in the field to develop a method for detecting and monitoring the information about health state of living organisms (e.g., humans, animals, plants, etc.), which has comparable sensitivity to trained animals but does not require individual training and extends beyond a service time of one animal. Additionally, a need exists for a method of deciphering the VC signature which can carry information about the health state of the organism.

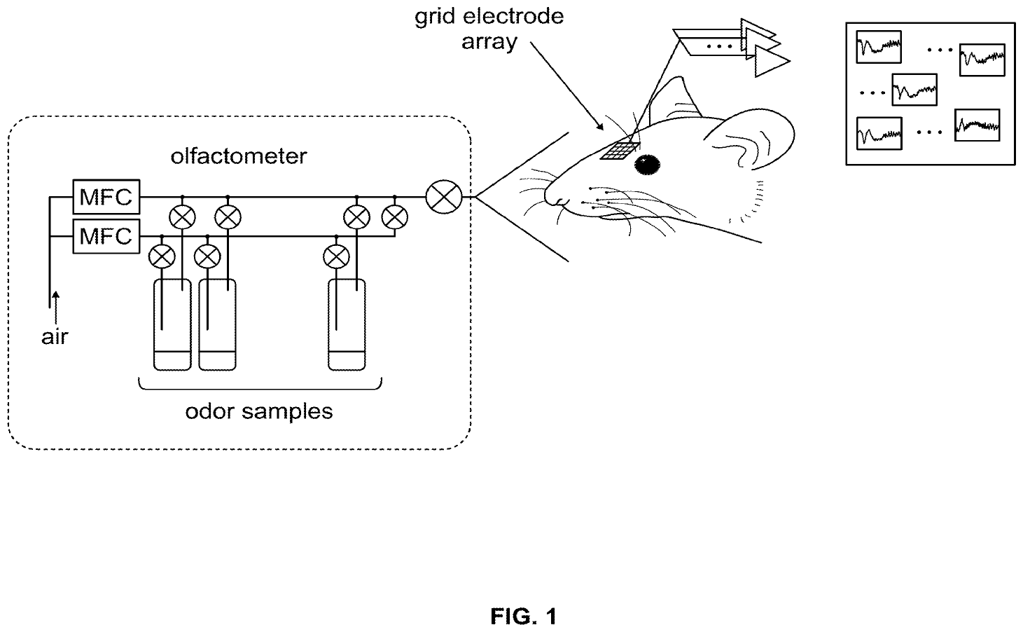

FIG. 1 illustrates a schematic of a brain machine interface (e.g., bio-electronic nose (BEN)). An animal (e.g., a rat) equipped with BMI can be presented with odor samples using an odor delivery device (e.g., olfactometer). The BMI can include a grid electrode array positioned on the surface of the olfactory bulb and connected to amplifiers and multiplexing chips. The electrode array can measure multi-channel raw signals. U.S. patent application Ser. No. 16/312,973 discusses a brain machine interface (e.g., bio-electronic nose), and is incorporated herein by reference.

In some embodiments, odorants may be presented to the service animal with the use of an odor delivery device. In some embodiments odorants may be presented to the service animal by direct access to the source of the odorant (e.g. a patient or biological sample from a patient

The early mammalian olfactory system can have the properties required by any chemical detector. The geometry of the nose and sniffing behavior can solve the non-trivial problem of fast (e.g., ˜100 ms) and reliable delivery of odorants to the chemical detectors as well as clearance (e.g., removal) of the odorants. These volatile odorants can bind to a subset of olfactory receptor (OR) types (e.g., rodents: ˜1200, K9: ˜900), each monoallelically expressed within the population of olfactory sensory neurons in the olfactory epithelium. The existence of a large number of different ORs can ensure high sensitivity to a broad range of different chemicals. All olfactory sensory neurons (OSNs) that express the same receptor can converge onto structures called glomeruli, which are arranged on the surface of the olfactory bulb. These glomeruli can integrate the signals from a large number of functionally identical sensors, thus maximizing the signal-to-noise ratio. The representation of chemical information at this level can be robust to animal learning or internal state. The signal from glomeruli can be further processed by bulbar neuronal network and sent to the cortex.

If a trained animal is capable of detecting a health state odorprint (e.g., disease odorprint), the disease-related information can be passed through its olfactory system. A BMI can extract this information and use it for disease detection.

The difficulty of identification of the disease odorprint, or odorprint of a health state in general, for animals including humans and plants, can be due to the large subject-to-subject variability. Thus, identification of the specific signature of the VC distribution carrying information about a health state of a living organism can require comparison of a large number of control and experimental subjects (e.g., disease carriers and healthy subjects). Even if it is possible to thoroughly analyze the VC distribution of individual subjects using analytical methods, such as gas-chromatography (GS) or gas chromatography combined with mass spectroscopy (GS-MS), it may be difficult to accumulate sufficient measurements which would allow statistical separation and identification of the signal of interest. During animal training, an animal can be exposed to a large number of samples and can slowly learn to identify the signal of interest. Intersecting the signal in the animal olfactory system using a BMI allows for searching (e.g., identifying, scanning, etc.) the differences between different states (e.g., healthy and diseased) without using animal training.

Once the signal carrying information about a health state of the organism is identified, it can be routinely used for monitoring a health state, for example to detect a specific disease (e.g., cancer). Furthermore, such a signal can be transferred to another animal (e.g., second animal) equipped with BMI using methods of transfer learning. The second animal can be used for health state assessment (e.g., disease diagnostics) without long training. See, for example, provisional application Ser. No. 63/220,361, incorporated herein by reference.

Grid electrode arrays can be used on the surface of the olfactory bulb to read odor information from the peripheral olfactory system. Such a grid electrode array can read the signal from the glomerulus layer. Each glomerulus can integrate and amplify the signal from thousands of olfactory sensory neurons expressing the same olfactory receptor type, and thus is sensitive to specific chemical features of presented volatile compounds. Spatio-temporal patterns of glomerulus activation can carry combinatorically rich information about presented odors, and relatively little affected by the behavioral state of the animal and higher cognitive processes.

The methods described herein can relate to alternative methods of reading glomerulus information. For example, these methods can include optical or acoustical methods. For example, glomerulus activity can be monitored using calcium, voltage or intrinsic imaging, or using acoustic or any other interfaces to the neural system. Additionally, odor related information can be read at a level other than the glomerulus level, for example using an interface to olfactory sensory neurons in the epithelium, the mitral/tufted cells, or cortical neurons.

In some embodiments, samples from multiple healthy and diseased subjects can be presented to a bio-electronic noses (e.g., a service animal with the brain machine interface to the olfactory system). The samples can include blood, blood plasma, urine, sweet, breath, or any other samples carrying information about a health state of the subject. Samples can be presented using an olfactometer. Each sample can evoke a complex pattern of neural activity recorded by the bio-electronic nose. Machine learning (ML) techniques, such as discriminant analysis, can be applied to find a difference between all plurality of samples from disease carrying subjects and control samples. The number of presented samples can include, for example, 2 samples, 5 samples, 10 samples, 50 samples, 100 samples, 200 samples, or more. The number of presented samples can be defined to be sufficient to find a statistically significant difference between diseased and control samples. The results of a discriminant analysis can be tested on the samples, which may not be used to train a discriminator. Discriminator analysis can allow for use for this analysis for the identification of future diseased samples (e.g., diagnostics).

In some embodiments, two bio-electronic noses can be synched by calibration against some number of generic odorants. The method can include measuring the response of the first bio-electronic nose to a new odorant outside the calibration set. This can allow for the prediction of a signature of this odorant on the second bio-electronic nose. The method can include recognizing this odorant using the second bio-electronic nose. The method can include projecting the discriminator to the second bio-electronic nose. The method can include performing identification of the disease samples using a bio-electronic nose which has not been previously trained on a large number of diseased and control samples. The information or data can be transferred from one bio-electronic nose to another bio-electronic nose. This approach opens a possibility of using many bio-electronic noses in parallel for diagnostics. This may be difficult to do with behaving animals, since each animal needs to be trained individually.

In some embodiments, the ability of the bio-electronic nose to identify disease samples can be used to decipher the chemical identity of volatile compounds and/or the quantitative composition of the VC mixture that is informative for disease identification. One of the samples of the diseased subject can be decomposed into individual volatile compounds by a means of mixture component separation based on physicochemical properties. In some embodiments, the separation can be achieved using a gas chromatography column. Individual components of the mixture can be delivered to an animal nose and their bio-electronic nose responses can be measured. The response of the original sample which constitutes a full mixture of all components and performance of the discriminator can be modeled by responses of individual volatile compounds. Those volatile compounds, which contribute mostly to the discriminator performance, can be considered as disease biomarkers. These volatile compounds can be identified using methods of analytical chemical analysis.

In some embodiments, the performance of the discriminator between diseased and control samples can be analyzed in the space of glomerulus spatiotemporal patterns. The glomeruli, which carry most of the information relevant for discriminator performance, can be considered as key discriminator glomeruli. One of the disease samples can be decomposed into individual volatile compounds using a gas chromatography column. Such a column can be split into two outputs. The first output can deliver the odor flow to an animal nose. The second output can include the gas ionization detector, mass spectrometer, or any other instrument for molecular identification. Those volatile compounds, which can evoke the activity of the key glomeruli in specific temporal sequence can be the ones which carry disease related information and are considered health state biomarkers. These volatile compound identities can be defined using the analytical instruments connected to the second capillary output.

In some embodiments, the bio-electronic nose can use a grid electrode array to record neural signals from the olfactory system. Diagnostic neural signals can also be extracted by other means, including but not limited to using optical, acoustic, or alternative electro-magnetic methods.

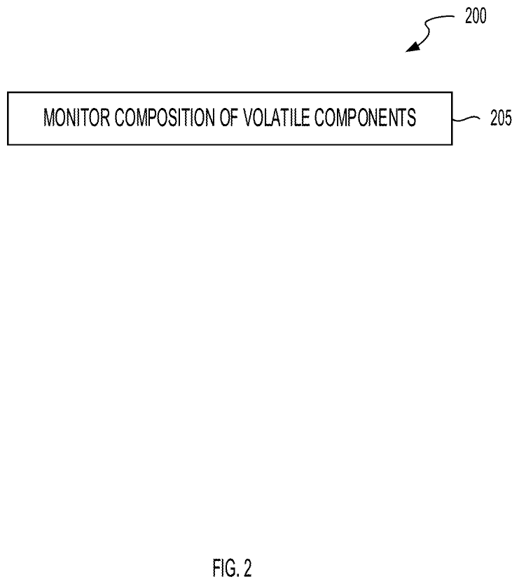

FIG. 2 illustrates a method 200 of identifying an odorprint of a health state of an organism. The method 200 can include monitoring a composition of volatile components (BLOCK 205). For example, the method 200 can include monitoring the composition of volatile components emitted by the organism. Monitoring the composition of volatile components emitted by the organism can be achieved using an olfactory system of a service animal. The service animal can be equipped with a brain machine interface. The brain machine interface can include an electrode assembly. The brain machine interface can include a chemical detector comprising an olfactory system in communication with the electrode assembly.

The method 200 can include identifying a signature of the health state corresponding to the composition of volatile compounds. The method 200 can include identifying the composition of volatile compounds emitted by the organism. The method 200 can include identifying a ratio of the volatile compounds emitted by the organism. The method 200 can include identifying a concentration of each of the volatile compounds emitted by the organism. The method 200 can include diagnosing a disease based on the composition of volatile components emitted by the organism. The organism can include at least one of a human, animal, or plant.

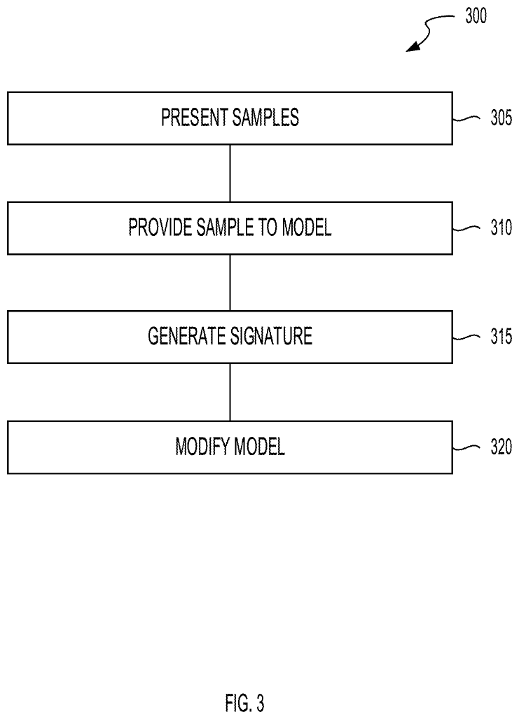

FIG. 3 illustrates a method of obtaining a signature of a brain machine interface. The method 300 can include presenting samples (BLOCK 305). For example, the method 300 can include presenting, by one or more processors, a plurality of samples from control subjects and experimental subjects. The control subjects and the experimental subjects can include at least one of humans, animals, or plants. The method 300 can include providing the samples to a model (BLOCK 310). For example, the method 300 can include providing, by the one or more processors, the presented samples to a machine learning model. The method 300 can include generating a signature (BLOCK 315). For example, the method 300 can include generating, by the machine learning model, a signature of a brain machine interface responsible for identification of a volatile compound odorprint based on multi-component brain machine interface signals. The brain machine interface can include an electrode assembly. The brain machine interface can include a chemical detector comprising an olfactory system in communication with the electrode assembly. The volatile compound odorprint can include a composition of volatile compounds emitted by the subjects. The method 300 can include modifying the model (BLOCK 320). For example, the method 300 can include modifying, by the one or more processors, the machine learning model based on the signature of the brain machine interface. The method 300 can include identifying a signature of a health state corresponding the volatile compound odorprint. The method 300 can include diagnosing a disease based on the volatile compound odorprint.

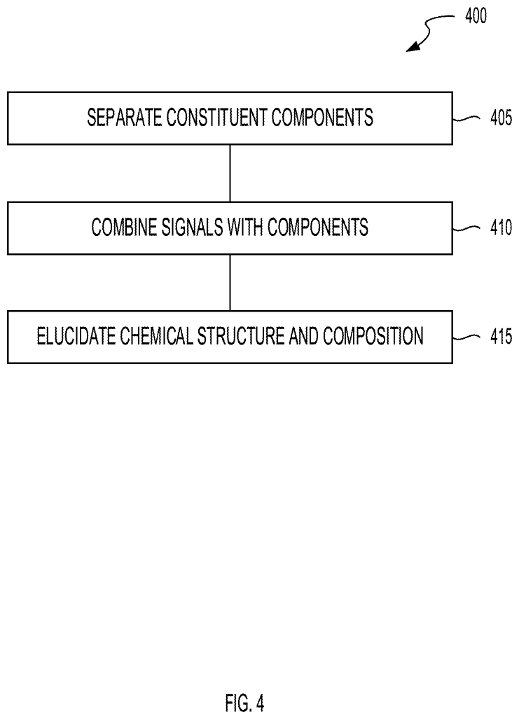

FIG. 4 illustrates a method of identifying volatile compounds. The method 400 can include separating constituent components (BLOCK 405). For example, the method 400 can include separating constituent components in mixtures based on physicochemical properties. The method 400 can include combining signals with components (BLOCK 410). For example, the method 400 can include combining brain machine interface signals with the separated constituent components. The brain machine interface signals can be generated from a brain machine interface. The brain machine interface can include an electrode assembly. The brain machine interface can include a chemical detector comprising an olfactory system in communication with the electrode assembly. The method 400 can include elucidating a chemical structure and a chemical composition (BLOCK 415). For example, the method 400 can include elucidating a chemical structure and a concentration of individual components based on the separated constituent components. The method 400 can include identifying the constituent components in the mixtures. The method 400 can include diagnosing a disease based on the constituent components in the mixtures. The method 400 can include identifying a signature of a health state corresponding the constituent components in the mixtures.

The subject matter and the operations described in this specification can be implemented in digital electronic circuitry, or in computer software, firmware, or hardware, including the structures disclosed in this specification and their structural equivalents, or in combinations of one or more of them. The subject matter described in this specification can be implemented as one or more computer programs, e.g., one or more circuits of computer program instructions, encoded on one or more computer storage media for execution by, or to control the operation of, data processing apparatus. Alternatively or in addition, the program instructions can be encoded on an artificially generated propagated signal, e.g., a machine-generated electrical, optical, or electromagnetic signal that can be generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium may not be a propagated signal, a computer storage medium can be a source or destination of computer program instructions encoded in an artificially generated propagated signal. The computer storage medium can also be, or be included in, one or more separate components or media (e.g., multiple CDs, disks, or other storage devices).

The operations described in this specification can be performed by a data processing apparatus on data stored on one or more computer-readable storage devices or received from other sources. The term “data processing apparatus” or “computing device” encompasses various apparatuses, devices, and machines for processing data, including by way of example a programmable processor, a computer, a system on a chip, or multiple ones, or combinations of the foregoing. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a circuit, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more circuits, subprograms, or portions of code). A computer program can be deployed to be executed on one computer or on multiple computers that are located at one site or distributed across multiple sites and interconnected by a communication network.

Processors suitable for the execution of a computer program include, by way of example, microprocessors, and any one or more processors of a digital computer. A processor can receive instructions and data from a read only memory or a random access memory or both. The elements of a computer are a processor for performing actions in accordance with instructions and one or more memory devices for storing instructions and data. A computer can include, or be operatively coupled to receive data from or transfer data to, or both, one or more mass storage devices for storing data, e.g., magnetic, magneto optical disks, or optical disks. A computer need not have such devices. Moreover, a computer can be embedded in another device, e.g., a personal digital assistant (PDA), a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

The implementations described herein can be implemented in any of numerous ways including, for example, using hardware, software or a combination thereof. When implemented in software, the software code can be executed on any suitable processor or collection of processors, whether provided in a single computer or distributed among multiple computers.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

A computer employed to implement at least a portion of the functionality described herein may comprise a memory, one or more processing units (also referred to herein simply as “processors”), one or more communication interfaces, one or more display units, and one or more user input devices. The memory may comprise any computer-readable media, and may store computer instructions (also referred to herein as “processor-executable instructions”) for implementing the various functionalities described herein. The processing unit(s) may be used to execute the instructions. The communication interface(s) may be coupled to a wired or wireless network, bus, or other communication means and may therefore allow the computer to transmit communications to or receive communications from other devices. The display unit(s) may be provided, for example, to allow a user to view various information in connection with execution of the instructions. The user input device(s) may be provided, for example, to allow the user to make manual adjustments, make selections, enter data or various other information, or interact in any of a variety of manners with the processor during execution of the instructions.

The various methods or processes outlined herein may be coded as software that is executable on one or more processors that employ any one of a variety of operating systems or platforms. Additionally, such software may be written using any of a number of suitable programming languages or programming or scripting tools, and also may be compiled as executable machine language code or intermediate code that is executed on a framework or virtual machine.

In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other non-transitory medium or tangible computer storage medium) encoded with one or more programs that, when executed on one or more computers or other processors, perform methods that implement features of the solution discussed above. The computer readable medium or media can be transportable, such that the program or programs stored thereon can be loaded onto one or more different computers or other processors to implement various aspects of the present solution as discussed above.

The terms “program” or “software” are used herein to refer to any type of computer code or set of computer-executable instructions that can be employed to program a computer or other processor to implement various aspects as discussed above. One or more computer programs that when executed perform methods of the present solution need not reside on a single computer or processor, but may be distributed in a modular fashion amongst a number of different computers or processors to implement various aspects of the present solution.

Computer-executable instructions may be in many forms, such as program modules, executed by one or more computers or other devices. Program modules can include routines, programs, objects, components, data structures, or other components that perform particular tasks or implement particular abstract data types. The functionality of the program modules can be combined or distributed as desired in various implementations.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

As used herein, the singular forms “a”, “an” and “the” include plural referents unless the context clearly dictates otherwise. Thus, for example, the term “a member” is intended to mean a single member or a combination of members, “a material” is intended to mean one or more materials, or a combination thereof.

As used herein, the terms “about” and “approximately” generally mean plus or minus 10% of the stated value. For example, about 0.5 would include 0.45 and 0.55, about 10 would include 9 to 11, about 1000 would include 900 to 1100.

It should be noted that the term “exemplary” as used herein to describe various embodiments is intended to indicate that such embodiments are possible examples, representations, and/or illustrations of possible embodiments (and such term is not intended to connote that such embodiments are necessarily extraordinary or superlative examples).

The terms “coupled,” “connected,” and the like as used herein mean the joining of two members directly or indirectly to one another. Such joining may be stationary (e.g., permanent) or moveable (e.g., removable or releasable). Such joining may be achieved with the two members or the two members and any additional intermediate members being integrally formed as a single unitary body with one another or with the two members or the two members and any additional intermediate members being attached to one another.

Any references to implementations or elements or acts of the systems and methods herein referred to in the singular can include implementations including a plurality of these elements, and any references in plural to any implementation or element or act herein can include implementations including only a single element. References in the singular or plural form are not intended to limit the presently disclosed systems or methods, their components, acts, or elements to single or plural configurations. References to any act or element being based on any information, act or element may include implementations where the act or element is based at least in part on any information, act, or element.

Any implementation disclosed herein may be combined with any other implementation, and references to “an implementation,” “some implementations,” “an alternate implementation,” “various implementations,” “one implementation” or the like are not necessarily mutually exclusive and are intended to indicate that a particular feature, structure, or characteristic described in connection with the implementation may be included in at least one implementation. Such terms as used herein are not necessarily all referring to the same implementation. Any implementation may be combined with any other implementation, inclusively or exclusively, in any manner consistent with the aspects and implementations disclosed herein.

References to “or” may be construed as inclusive so that any terms described using “or” may indicate any of a single, more than one, and all of the described terms. References to at least one of a conjunctive list of terms may be construed as an inclusive OR to indicate any of a single, more than one, and all of the described terms. For example, a reference to “at least one of ‘A’ and ‘B’” can include only ‘A’, only ‘B’, as well as both ‘A’ and ‘B’. Elements other than ‘A’ and ‘B’ can also be included.

The systems and methods described herein may be embodied in other specific forms without departing from the characteristics thereof. The foregoing implementations are illustrative rather than limiting of the described systems and methods.

Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any claim elements.

The systems and methods described herein may be embodied in other specific forms without departing from the characteristics thereof. The foregoing implementations are illustrative rather than limiting of the described systems and methods. Scope of the systems and methods described herein is thus indicated by the appended claims, rather than the foregoing description, and changes that come within the meaning and range of equivalency of the claims are embraced therein.

While this specification contains many specific implementation details, these should not be construed as limitations on the scope of any inventions or of what may be claimed, but rather as descriptions of features specific to particular implementations of particular inventions. Certain features described in this specification in the context of separate implementations can also be implemented in combination in a single implementation. Conversely, various features described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

What is claimed is:

1. A method of identifying an odorprint of a health state of an organism, comprising:
monitoring a composition of volatile components emitted by the organism using an olfactory system of a service animal, the service animal equipped with a brain machine interface.

2. The method of claim 1, further comprising identifying a signature of the health state corresponding to the composition of volatile compounds.

3. The method of claim 1, further comprising identifying the composition of volatile compounds emitted by the organism.

4. The method of claim 1, further comprising identifying a ratio of the volatile compounds emitted by the organism.

5. The method of claim 1, wherein the brain machine interface comprises:
an electrode assembly; and
a chemical detector comprising an olfactory system in communication with the electrode assembly.

6. The method of claim 1, further comprising identifying a concentration of each of the volatile compounds emitted by the organism.

7. The method of claim 1, further comprising diagnosing a disease based on the composition of volatile components emitted by the organism.

8. The method of claim 1, wherein the organism comprises at least one of a human, animal, or plant.

9. A method of obtaining a signature of a brain machine interface, comprising:
presenting, by one or more processors, a plurality of samples from control subjects and experimental subjects;
providing, by the one or more processors, the presented samples to a machine learning model;
generating, by the machine learning model, the signature of a brain machine interface responsible for identification of a volatile compound odorprint based on multi-component brain machine interface signals; and
modifying, by the one or more processors, the machine learning model based on the signature of the brain machine interface.

10. The method of claim 9, wherein the volatile compound odorprint comprises a composition of volatile compounds emitted by the subjects.

11. The method of claim 9, wherein the brain machine interface comprises:
an electrode assembly; and
a chemical detector comprising an olfactory system in communication with the electrode assembly.

12. The method of claim 9, further comprising identifying a signature of a health state corresponding the volatile compound odorprint.

13. The method of claim 9, further comprising diagnosing a disease based on the volatile compound odorprint.

14. The method of claim 9, wherein the control subjects and the experimental subjects comprise at least one of humans, animals, or plants.

15. A method of identifying volatile compounds, comprising:
separating constituent components in mixtures based on physicochemical properties;
combining brain machine interface signals with the separated constituent components; and
elucidating a chemical structure and a concentration of individual components based on the separated constituent components.

16. The method of claim 15, wherein the brain machine interface signals are generated from a brain machine interface.

17. The method of claim 15, wherein the brain machine interface signals are generated from a brain machine interface comprising:
an electrode assembly; and
a chemical detector comprising an olfactory system in communication with the electrode assembly.

18. The method of claim 15, further comprising identifying the constituent components in the mixtures.

19. The method of claim 15, further comprising diagnosing a disease based on the constituent components in the mixtures

20. The method of claim 15, further comprising identifying a signature of a health state corresponding the constituent components in the mixtures.
